(19) United States
(12) Patent Application Publication
(10) Pub. No.: US 2011/0264000 A1
(43) Pub. Date: Oct. 27, 2011

(54) SYSTEM AND METHOD FOR DETERMINING TISSUE TYPE AND MAPPING TISSUE MORPHOLOGY

(76) Inventors: SAURAV PAUL, MINNEAPOLIS, MN (US); PRATHYUSHA MARRI, MINNEAPOLIS, MN (US); D. CURTIS DENO, ANDOVER, MN (US)

(21) Appl. No.: 13/102,002

(22) Filed: May 5, 2011

Related U.S. Application Data
(63) Continuation-in-part of application No. 11/966,320, filed on Dec. 28, 2007.
Application No. 11/966,320 is a continuation-in-part of application No. 12/964,910, filed on Dec. 10, 2010.
Application No. 12/964,910 is a continuation-in-part of application No. 12/946,941, filed on Nov. 16, 2010.
Application No. 12/946,941 is a continuation-in-part of application No. 12/622,488, filed on Nov. 20, 2009.
(60) Provisional application No. 61/177,876, filed on May 13, 2009.

(51) Int. Cl. A61B 5/053

(52) U.S. Cl. 600/547

(57) ABSTRACT

A method and system for determining tissue type is provided. The system comprises an electronic control unit (ECU) configured to acquire a value of an electrical parameter between a first electrode electrically coupled with tissue and a second electrode. The ECU is further configured to identify a tissue type from a plurality of tissue types based at least on the acquired value, and in an exemplary embodiment, to generate a tissue morphology map comprising a marker representative of the identified tissue type. The method comprises acquiring a value of an electrical parameter between a first electrode electrically coupled with tissue and a second electrode. The method further comprises identifying a tissue type from a plurality of tissue types based on at least the acquired value of the electrical parameter, and in an exemplary embodiment, the method further comprises generating a tissue morphology map based on the identified tissue type.

### CROSS REFERENCE TO RELATED APPLICATIONS

This application is a continuation-in-part of U.S. patent application Ser. No. 11/966,320 filed Dec. 28, 2007 and currently pending. This application is also a continuation-in-part of U.S. patent application Ser. No. 12/964,910 filed Dec. 10, 2010 and currently pending, which, in turn, is a continuation-in-part of U.S. patent application Ser. No. 12/946,941 filed Nov. 16, 2010 and currently pending, which, in turn, is a continuation-in-part of U.S. patent application Ser. No. 12/622,488 filed Nov. 20, 2009 and currently pending, which, in turn, claims the benefit of U.S. Provisional Patent Application Ser. No. 61/177,876 filed May 13, 2009, and now expired. The disclosures of each of the above identified applications are hereby incorporated by reference in their entireties.

### BACKGROUND OF THE INVENTION

a. Field of the Invention

This disclosure relates to a system and method for determining tissue type. More particularly, this disclosure relates to a system and method for determining or identifying a tissue type for a location in tissue, and generating a tissue morphology map based on the identified tissue type.

b. Background Art

It is known that ablation therapy may be used to treat various conditions afflicting the human anatomy. One such condition that ablation therapy finds particular applicability is in the treatment of atrial arrhythmias, for example. When tissue is ablated, or at least subjected to ablative energy generated by an ablation generator and delivered by an ablation catheter, lesions form in the tissue. More particularly, an electrode or electrodes mounted on or in the ablation catheter are used to create tissue necrosis in cardiac tissue to correct conditions such as atrial arrhythmia (including, but not limited to, ectopic atrial tachycardia, atrial fibrillation, and atrial flutter). Atrial arrhythmias can create a variety of dangerous conditions including irregular heart rates, loss of synchronous atrioventricular contractions and stasis of blood flow which can lead to a variety of ailments and even death. It is believed that the primary cause of atrial arrhythmia is stray electrical signals within the left or right atrium of the heart. The ablation catheter imparts ablative energy (e.g., radio frequency energy, cryoablation, lasers, chemicals, high-intensity focused ultrasound, etc.) to cardiac tissue to create a lesion in the cardiac tissue. The lesion disrupts undesirable electrical pathways and thereby limits or prevents stray electrical signals that lead to arrhythmias.

One challenge with ablation procedures is in the assessment or determination of the tissue type or morphology of the tissue in and around an ablation site. For example, it may be difficult to determine whether a particular area of tissue is scar tissue, fat tissue, ablated or lesioned tissue, or regular tissue (e.g., endocardial, myocardial, or epicardial tissue). Accordingly, because it has been difficult to distinguish one type of tissue from another, it is also difficult to determine which areas of tissue ablative energy should be applied to during an ablation procedure. For example, a clinician may wish to apply ablative energy to regular tissue and avoid the application of such energy to fat tissue because efficacious lesions cannot be created in fat tissue. In another example, a clinician may wish to ablate on the border between scar and regular tissue to isolate the scar tissue that triggers ventricular tachycardia. In either instance, if the clinician cannot distinguish between the different tissue types, the ablation procedure cannot be performed in the most efficient manner possible.

Conventional techniques used to determine tissue type or morphology include, for example and without limitation, ultrasound, magnetic resonance, or microwave-based imaging modalities. While these techniques provide images of the tissue and may allow for a clinician to discern tissue type for a particular location in the tissue, they are not without their drawbacks. For instance, each of the aforementioned conventional techniques require wholly separate systems from the ablation system and/or the visualization, navigation, and mapping system typically used therewith. As a result, additional components are required to carry out the functionality of determining tissue type, thereby increasing, among other things, the complexity of the overall system and the cost of the procedure.

Accordingly, the inventors herein have recognized a need for a system and method for determining tissue type that will minimize and/or eliminate one or more of the deficiencies in conventional systems.

### BRIEF SUMMARY OF THE INVENTION

The present invention is directed to a system and method for determining tissue type. The system, in accordance with present teachings, comprises an electronic control unit (ECU) configured to acquire a value of an electrical parameter between a first electrode in electrically coupled with tissue and a second electrode. The ECU is further configured to identify a tissue type from a plurality of tissue types based on at least the acquired value of the electrical parameter. In an exemplary embodiment, the ECU is still further configured to generate a tissue morphology map comprising a marker representative of the identified tissue type.

In accordance with another aspect of the invention, a method of determining tissue type is provided. In accordance with the present teachings, the method includes a step of acquiring a value of an electrical parameter between a first electrode electrically coupled with tissue and a second electrode. The method further comprises a step of identifying a tissue type from a plurality of tissue types based on at least the acquired value of the electrical parameter. In an exemplary embodiment, the method still further comprises a step of generating a tissue morphology map based on the identified tissue type.

In accordance with yet another aspect of the invention, a method of presenting information representative of determined tissue type is provided. In accordance with the present teachings, the method includes a step of acquiring a value of an electrical parameter between a first electrode electrically coupled with tissue and a second electrode. The method further comprises a step of identifying a tissue type from a plurality of tissue types based on at least the acquired value of the electrical parameter. The method still further comprises a step of determining a location in the tissue corresponding to the acquired value based on a position of the first electrode. The method yet still further comprises a step of generating a marker representative of the identified tissue type; and a step of superimposing the marker onto a portion of an image or model of the tissue corresponding to the determined location.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 is a schematic and diagrammatic view of a system for determining tissue type and generating a tissue morphology map based on determined tissue type in accordance with the present teachings.

FIGS. 2a-2c are diagrammatic views of exemplary embodiments of the distal portion of the catheter illustrated in FIG. 1.

FIGS. 3a-3e are partial simplified schematic and diagrammatic views of exemplary embodiments of the system illustrated in FIG. 1.

FIG. 4 is a simplified schematic and diagrammatic view of an exemplary embodiment of the visualization, navigation, and mapping system of the system illustrated in FIG. 1.

FIG. 5 is an exemplary embodiment of a display device of the system illustrated in FIG. 1 with a graphical user interface (GUI) displayed thereon.

FIG. 6 is flow chart illustrative of an exemplary embodiment of a method for determining tissue type in accordance with the present teachings.

FIG. 7 is a table showing an exemplary embodiment of how data acquired by the system of FIG. 1 is organized and/or stored.

FIG. 8 is a schematic and diagrammatic view of a portion of the system illustrated in FIG. 1 used in connection with time-dependent gating.

### DETAILED DESCRIPTION OF EMBODIMENTS OF THE INVENTION

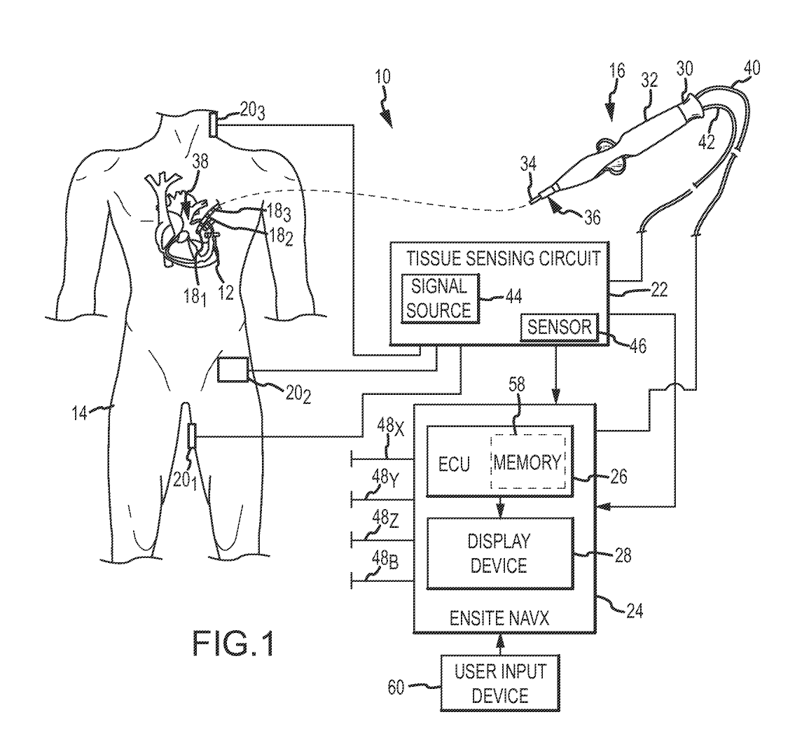

Referring now to the drawings wherein like reference numerals are used to identify identical components in the various views, FIG. 1 illustrates one exemplary embodiment of a system 10 for performing one more diagnostic and/or therapeutic functions. More particularly, the system 10 includes components for, among other things, determining tissue type for a tissue 12 of a body 14 and, in an exemplary embodiment, mapping tissue morphology based on determined tissue type. In an exemplary embodiment, the tissue 12 comprises heart or cardiac tissue within a human body 14. It should be understood, however, that the system 10 may find application in connection with a variety of other tissues within human and non-human bodies.

Among other components, the system 10 includes a medical device, such as, for example, a catheter 16 having, as will be described in greater detail below, one or more electrodes 18 mounted thereon. The system 10 may further include one or more patch electrodes, such as, for example, patch electrodes 20 (e.g., electrodes 20₁, 20₂, 20₃), a tissue sensing circuit 22, and a system 24 for the visualization, navigation, and/or mapping of internal body structures, which may include, for example and without limitation, an electronic control unit (ECU) 26 and a display device 28. Alternatively, the ECU 26 and/or the display 28 may be separate and distinct from, but electrically connected to and configured for communication with, the system 24.

With continued reference to FIG. 1, the catheter 16 is provided for examination, diagnosis, and/or treatment of internal body tissues such as the tissue 12. In an exemplary embodiment, the catheter 16 comprises an ablation catheter, such as, for example, an irrigated radio-frequency (RF) ablation catheter. It should be understood, however, that catheter 16 is not limited to an irrigated and/or RF-based ablation catheter, or to an ablation catheter at all. Rather, in other exemplary embodiments, the catheter 16 may comprise a non-irrigated catheter and/or other types of ablation catheters (e.g., cryoablation, ultrasound, etc.), or other therapeutic and/or diagnostic catheters.

The catheter 16 may include a cable connector or interface 30, a handle 32, a shaft 34 having a proximal end 36 and a distal end 38 (as used herein, “proximal” refers to a direction toward the end of the catheter 16 near the clinician, and “distal” refers to a direction away from the clinician and (generally) inside the body of a patient), and one or more electrodes, such as for example and as will be described in greater detail below, electrodes 18 (i.e., 18₁, 18₂, . . . ,18_{N}) mounted in or on the shaft 34 of the catheter 16. In an exemplary embodiment, the electrodes 18 are disposed at or near the distal end 38 of the shaft 34. The catheter 16 may further include other conventional components such as, for example and without limitation, a temperature sensor, additional electrodes, ablation elements (e.g., ablation tip electrodes for delivering RF ablative energy, high intensity focused ultrasound ablation elements, etc.), and corresponding conductors or leads.

The connector 30 provides mechanical, fluid, and electrical connection(s) for cables, such as, for example, cables 40, 42 extending to the tissue sensing circuit 22, the visualization, navigation, and/or mapping system 24, and other components of the system 10 (e.g., an ablation generator, irrigation source, etc.). The connector 30 is conventional in the art and is disposed at the proximal end 36 of the catheter 16.

The handle 32 provides a location for the clinician to hold the catheter 16 and may further provide means for steering or guiding the shaft 34 within the body 14. For example, the handle 32 may include means to change the length of a steering wire extending through the catheter 16 to the distal end 38 of the shaft 34 to steer the shaft 34. The handle 32 is also conventional in the art and it will be understood that the construction of the handle 32 may vary. In another exemplary embodiment, the catheter 16 may be robotically driven or controlled. Accordingly, rather than a clinician manipulating a handle to steer or guide the catheter 16, and the shaft 34 thereof, in particular, a robot is used to manipulate the catheter 16.

The shaft 34 is an elongate, tubular, flexible member configured for movement within the body 14. The shaft 34 supports, for example and without limitation, electrodes mounted thereon, such as, for example, the electrodes 18, associated conductors, and possibly additional electronics used for signal processing or conditioning. The shaft 34 may also permit transport, delivery and/or removal of fluids (including irrigation fluids, cryogenic ablation fluids, and bodily fluids), medicines, and/or surgical tools or instruments. The shaft 34 may be made from conventional materials such as polyurethane, and defines one or more lumens configured to house and/or transport electrical conductors, fluids, or surgical tools. The shaft 34 may be introduced into a blood vessel or other structure within the body 14 through a conventional introducer. The shaft 34 may then be steered or guided through the body 14 to a desired location, such as the tissue 12, using means well known in the art.

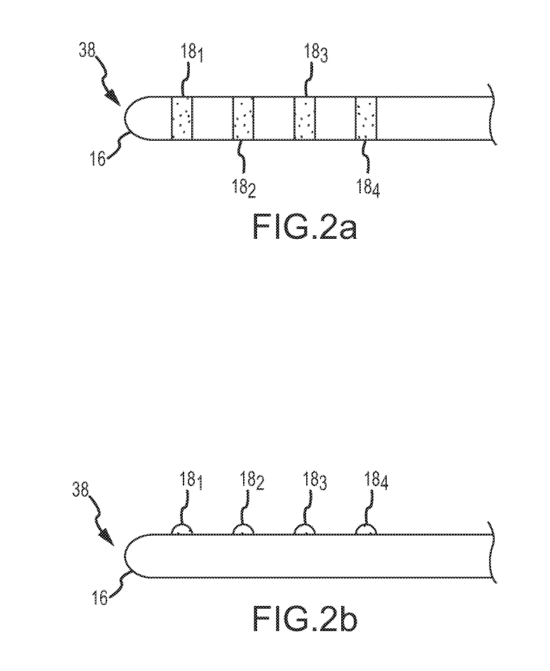

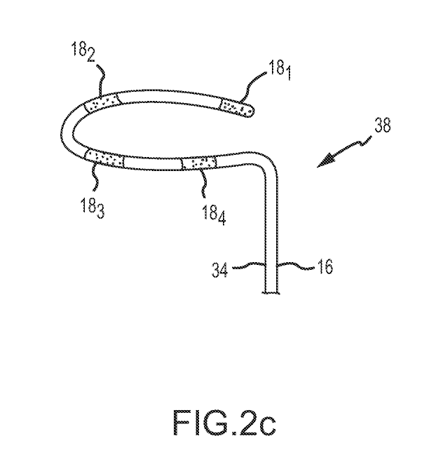

The electrodes 18 mounted on the shaft 34 of the catheter 16 are provided for a variety of diagnostic and therapeutic purposes including, for example, electrophysiological studies, catheter identification and location, pacing, cardiac mapping, and ablation. In the illustrated embodiment, the catheter 16 includes a plurality of electrodes 18, such as electrodes 18₁, 18₂, 18₃, 18₄, disposed in or on the shaft 34 near the distal end 38 thereof, some or all of which are electrically connected to the tissue sensing circuit 22 and/or other components of the system 10 (e.g., ablation generator, visualization, navigation, and mapping system 24, etc.). The electrodes 18 may comprise one of a number of types of electrodes, such as, for example and without limitation, tip electrodes (See FIG. 1), ring electrodes (See FIG. 2a), button electrodes (See FIG. 2b), coil electrodes, brush electrodes, flexible polymer electrodes, and spot electrodes. It will be appreciated that while only certain embodiments of the catheter 16 having particular numbers and types of electrodes mounted therein or thereon are described in detail herein, the number, shape, orientation, and purpose of the electrodes may vary, and embodiments wherein the catheter 16 has electrodes different than those specifically described herein remain within the spirit and scope of the present disclosure.

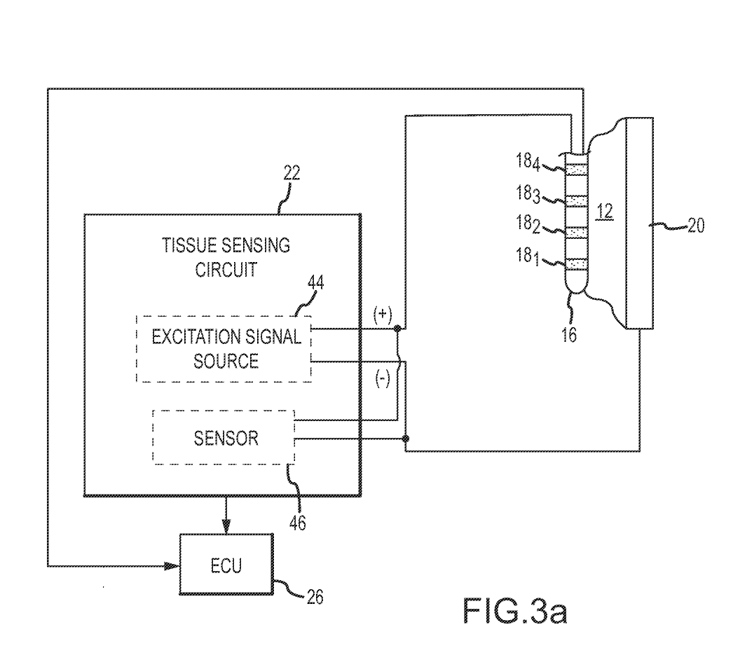

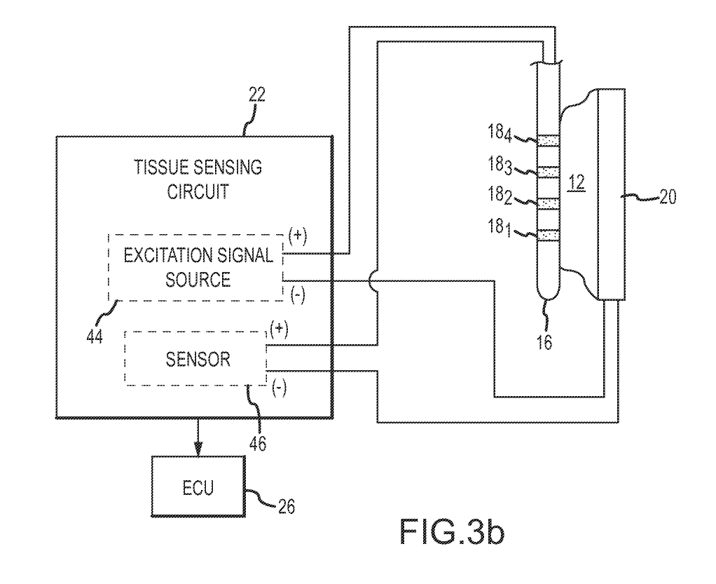

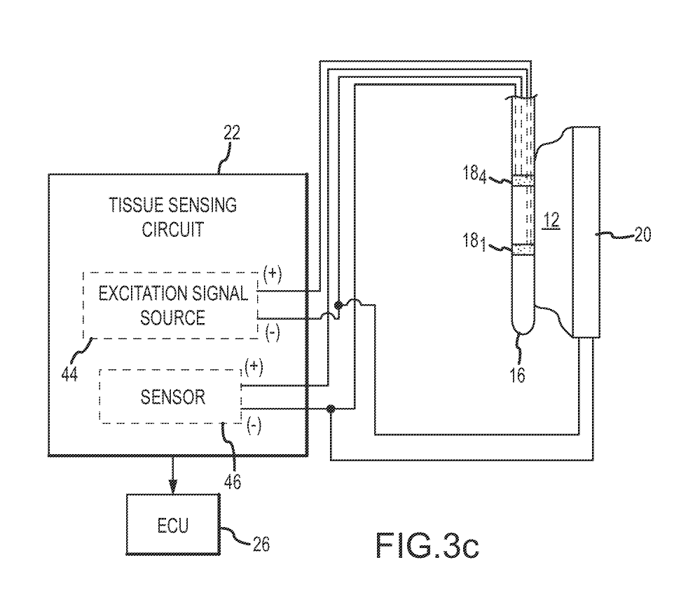

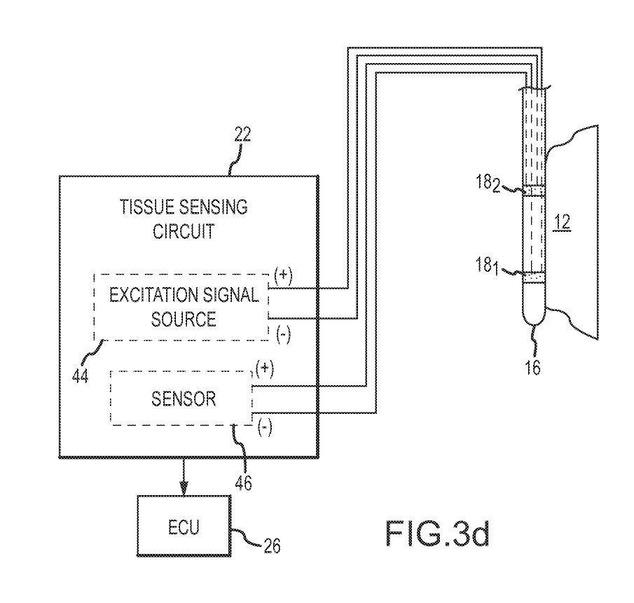

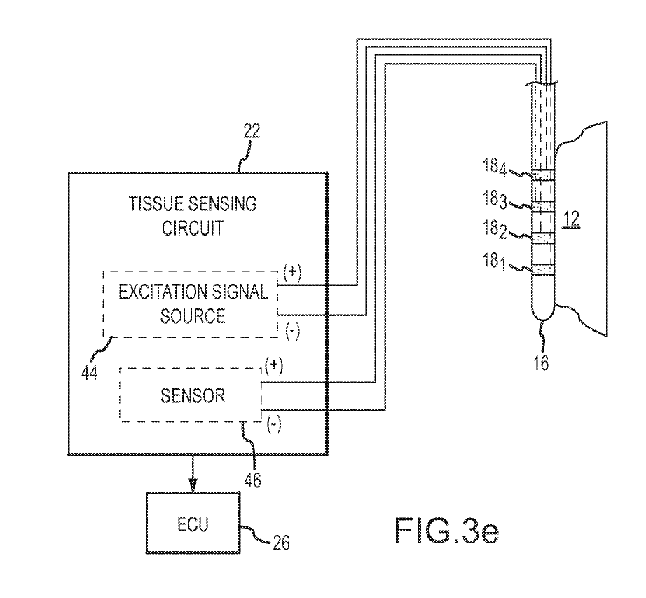

With reference to FIGS. 3a-3e, the tissue sensing circuit 22 is configured to measure one or more electrical parameters, properties, or attributes relating to the tissue 12. In an exemplary embodiment, the tissue sensing circuit 22 provides a means, such as a tissue sensing signal source 44, for generating an excitation signal used in the measurement of one or more electrical parameters, and means, such as a sensor 46, for sensing values of the electrical parameter(s). For example in an exemplary embodiment, the signal source 44 is configured for generating an excitation signal used in complex impedance measurements, and the sensor 46 is configured to resolve the determined impedance into its component parts. The signal source 44 is coupled, effectively, across two or more electrodes. In one exemplary embodiment, a unipolar measurement mode or technique is used wherein the signal source is effectively coupled across at least one of the electrodes 18 mounted in or on the catheter 16 and a dispersive/indifferent reference patch electrode 20 configured to be affixed to the body 14 and in electrical contact with the patient's skin (See FIGS. 3a-3c, for example). Alternatively, in another exemplary embodiment, a bipolar measurement mode or technique is used wherein the signal source 44 is effectively coupled across two or more of the electrodes 18 (e.g., 18₁, 18₂) mounted in or on the catheter 16, or at least one of the electrodes 18 of the catheter 16 and an electrode mounted on another catheter used in conjunction with the catheter 16 (See FIGS. 3d and 3e, for example).

Whether a unipolar or bipolar measurement mode is utilized, the signal source 44 is configured, as was briefly described above, to generate the excitation signal used in determining values of one or more electrical parameters, such as, for example and without limitation, the complex impedance between the electrodes. More particularly, the excitation signal, when applied across the electrodes, will produce a corresponding induced signal whose characteristics will be determined by, and thus be indicative of, the properties of the tissue under test. In one embodiment, the excitation signal has a predetermined frequency within a range from about 20 kHz to 1000 kHz, and more preferably within a range of about 50 kHz to 500 kHz. In another exemplary embodiment, and as will be described in greater detail below, the signal source 44 may be configured to generate excitation signals of different frequencies, as opposed to only generating excitation signals of a single frequency. In still another exemplary embodiment that will also be described in greater detail below, the signal source 44 may be additionally or alternatively configured to generate a burst signal with a spectrum of frequencies as opposed to a discrete frequency. In any of these embodiments, the signal source 44 may comprise conventional apparatus for generating such signals (e.g., may be either a constant voltage or current at the predetermined frequency).

As with the signal source 44 described above, in an exemplary embodiment, the sensor 46 is coupled, effectively, across two or more electrodes. In an exemplary embodiment, the electrodes across which the sensor 46 is effectively coupled are the same electrodes across which the signal source 44 is effectively coupled. In another exemplary embodiment, however, the sensor 46 is not effectively coupled across the same two electrodes across which the signal source 44 is coupled. For example, the sensor 46 may be effectively coupled across one of the electrodes coupled to the signal source 44 and another electrode not coupled to the signal source 44. In another embodiment, the sensor 46 is effectively coupled across two or more electrodes that are not also coupled to the signal source 44. Further, as illustrated in FIG. 3a, like terminals (e.g., the positive (+) or negative (−) terminals) of the signal source 44 and the sensor 46 may be coupled together and connected to the same electrodes with a single wire. Accordingly, the positive terminals of the signal source 44 and the sensor 46 may be coupled together and electrically connected to an electrode with a single wire, and the negative terminals of the signal source 44 and the sensor 46 may be coupled together and electrically connected to an electrode other than the electrode to which the positive terminals are connected with a single wire. Alternatively, as illustrated in FIGS. 3b and 3c, in another exemplary embodiment, each of the terminals of the signal source 44 and the sensor 46 may be coupled to electrodes (whether or not like terminals of the signal source 44 and the sensor 46 are coupled to common electrodes) using separate wires (e.g., 4 or more wires extending between terminals and electrodes). Accordingly, each terminal of the signal source and the sensor 46 are coupled to an electrode with a dedicated wire.

In any event, in an exemplary embodiment the sensor 46 is configured to determine a complex impedance between the electrodes, based on the measurement of the induced signal, in view of the applied excitation signal. More particularly, the sensor 46 is configured to resolve the complex impedance into its component parts (i.e., the resistance (R) and reactance (X) or the impedance magnitude (|Z|) and phase angle (∠Z or φ)).

In an embodiment wherein the unipolar mode is utilized (i.e., the electrodes across which the sensor 46 is effectively coupled comprise at least one of the electrodes 18 of the catheter 16 and a reference patch electrode 20), the complex impedance measurement reflects the properties of the tissue near and around the electrode 18. For example, the sensor 46 may be effectively coupled across electrode 18₁ and one of patch electrodes 20 (e.g., patch electrodes 20₁, 20₂, or 20₃ illustrated in FIG. 1) and the measurement reflects the properties of the tissue near and around the electrode 18₁. Likewise, the sensor 46 may also (simultaneously or in a gated fashion) take measurements across electrode 18₂ and one of patch electrodes 20 (e.g., patch electrodes 20₁, 20₂, or 20₃ illustrated in FIG. 1).

In an embodiment wherein the bipolar mode is utilized (i.e., the electrodes across which the sensor 46 is effectively coupled comprise two or more of the electrodes 18 of the catheter 16, or one or more of the electrodes 18 of the catheter 16 and an electrode of another catheter), the complex impedance measurement reflects the properties of the tissue disposed between the electrodes. For example, in an exemplary embodiment wherein the electrodes 18 are arranged in a substantially straight line (See, for example, FIGS. 2A and 2B), the signal source 44 and the sensor 46 may both be effectively coupled across any two of the electrodes 18₁-18₄, and the measurement reflects the properties of the tissue disposed between those two electrodes. In the embodiment illustrated in FIG. 3d, for example, the signal source 44 and the sensor 46 are coupled across electrodes 18₁ and 18₂. In another exemplary, the signal source 44 may be effectively coupled across two of the electrodes 18, and the sensor 46 may be coupled across either one of the electrodes coupled to the signal source 44 and an electrode disposed between the two electrodes across which the source 44 is coupled, or two electrodes disposed between the two electrodes across which the source 44 is coupled. For example, in one embodiment illustrated in FIG. 3e and provided for exemplary purposes only, the signal source 44 is coupled across electrodes 18₁ and 18₄, and the sensor 46 is coupled across electrode pair 18₂ and 18₃. In other exemplary embodiments, the sensor 46 may be alternatively or additionally coupled across one or more of the electrode pairs 18₁ and 18₂, 18₁ and 18₃, 18₂ and 18₄, and 18₃ and 18₄. In such an embodiment, the measurement reflects the properties of the tissue disposed between the two electrodes across which the sensor 46 is effectively coupled.

In another exemplary embodiment wherein the bipolar mode is utilized, rather than the electrodes 18 being arranged in a linear fashion as illustrated in FIGS. 2A and 2B, the electrodes are arranged so as to form a polygonal shape, such as, for example and without limitation, a rectangle or square. In such an embodiment, the catheter 16 may be a spiral catheter such as that illustrated in FIG. 2c, or may comprise a catheter such as that illustrated in FIGS. 2A and 2B but with the electrodes 18₁-18₄ arranged, for example and as is well known in the art, in a four point arrangement. In any event, the signal source 44 is effectively coupled across diagonal electrodes (e.g., electrodes 18₁₁ and 18₃, or 18₂ and 18₄, in FIG. 2c), while the sensor 46 is effectively coupled across the same diagonal electrodes across which the signal source 44 is coupled, or the other diagonally arranged electrode pair not coupled to the signal source 44 (e.g., the sensor 46 is coupled across electrodes 18₂ and 18₄ if the signal source 44 is coupled across electrodes 18₁ and 18₃, and across electrodes 18₁ and 18₃ if the signal source 44 is coupled across the electrodes 18₂ and 18₄). As with the embodiments described above, the measurement reflects the properties of the tissue disposed between the two electrodes across which the sensor 46 is effectively coupled.

Whether a unipolar or bipolar measurement mode is utilized, sensor 46, which may comprise conventional apparatus for performing such measurements and processing, in view of the selected nature and format of the applied excitation signal, may include conventional filters (e.g., bandpass filters) to block frequencies that are not of interest, but permit appropriate frequencies, such as the excitation frequency, to pass, as well as conventional signal processing software used to obtain, for example, the component parts of the measured complex impedance. In an exemplary embodiment, the signal source 44 and the sensor 46 may be integrated (e.g., like an LCR meter).

In an exemplary embodiment wherein the unipolar measuring mode is utilized, the measurements may be with respect to a single electrode 18 and the dispersive/indifferent electrode 20 (e.g., one of electrodes 20₁, 20₂, 20₃ illustrated in FIG. 1, for example). However, in another exemplary embodiment, a “multi-unipolar mode” may be employed wherein measurements between multiple electrodes 18 and the dispersive/indifferent electrode 20, or multiple dispersive/indifferent electrodes, may be made substantially simultaneously. In such an embodiment, the excitation signal source 44 and sensor 46 would both be effectively coupled across each of the electrodes 18 and/or one or more dispersive/indifferent electrodes 20, and each of the complex impedance measurements would reflect the properties of the tissue near and around the respective electrode 18 to which the measurement corresponds. Accordingly, complex impedance measurements can be acquired for multiple locations in the tissue 12 at the same time.

Similarly, in an exemplary embodiment wherein the bipolar measuring mode is utilized, the measurements may be with respect to a single pair of electrodes, such as, for example, electrodes 18₁ and 18₂. However, in another exemplary embodiment, a “multi-bipolar mode” may be employed wherein measurements between a plurality of electrode pairs may be made simultaneously. For example, measurements between electrodes 18₁ and 18₂ may be made simultaneously with measurements between other electrode pairs, such as, for example, electrodes 18₁ and 18₃, electrodes 18₁ and 18₄, electrodes 18₂ and 18₃, electrodes 18₂ and 18₄, and/or electrodes 18₃ and 18₄. In such an embodiment, the sensor 46 would be effectively coupled across each electrode pair, and each of the complex impedance measurements would reflect the properties of the tissue between the electrodes 18 of the respective electrode pairs to which the measurements correspond. Accordingly, and as with the multi-unipolar mode described above, complex impedance measurements can be acquired for multiple locations in the tissue 12 at the same time.

It will be appreciated that while the description above and below is primarily limited to an embodiment wherein the electrical parameter measured by the tissue sensing circuit 22 comprises the complex impedance, and the components thereof, in particular, in other exemplary embodiments, such as those described below, other electrical parameters known in the art may be measured and used as described herein. Accordingly, embodiments of the system 10 having a tissue sensing circuit 22 that measures electrical parameters other than complex impedance remain within the spirit and scope of the present disclosure. For purposes of clarity and illustration only, however, the description below will be primarily with respect to an embodiment wherein the electrical parameter(s) comprises complex impedance and/or the components thereof.

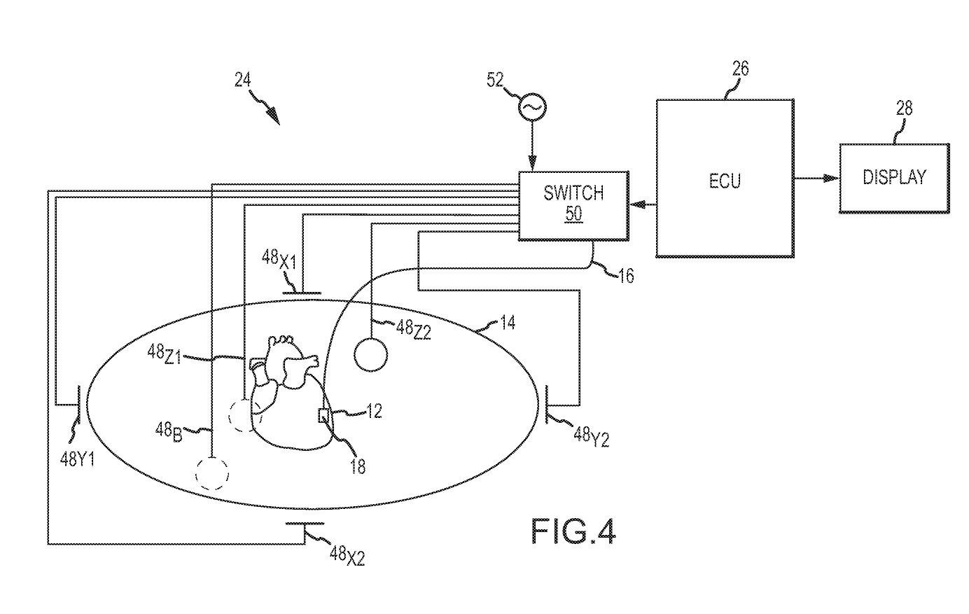

With reference to FIGS. 1 and 4, the visualization, navigation, and mapping system 24 will be described. The system 24 is provided for visualization, navigation, and/or mapping of internal body structures. The visualization, navigation, and/or mapping system 24 may comprise an electric field-based system, such as, for example, that having the model name EnSite NavX™ and commercially available from St. Jude Medical., Inc. and as generally shown with reference to U.S. Pat. No. 7,263,397 titled “Method and Apparatus for Catheter Navigation and Location and Mapping in the Heart,” the entire disclosure of which is incorporated herein by reference. In other exemplary embodiments, however, the visualization, navigation, and/or mapping system may comprise other types of systems, such as, for example and without limitation: a magnetic-field based system such as the Carto™ System available from Biosense Webster, and as generally shown with reference to one or more of U.S. Pat. No. 6,498,944 entitled “Intrabody Measurement,” U.S. Pat. No. 6,788,967 entitled “Medical Diagnosis, Treatment and Imaging Systems,” and U.S. Pat. No. 6,690,963 entitled “System and Method for Determining the Location and Orientation of an Invasive Medical Instrument,” the entire disclosures of which are incorporated herein by reference, or the gMPS system from MediGuide Ltd., and as generally shown with reference to one or more of U.S. Pat. No. 6,233,476 entitled “Medical Positioning System,” U.S. Pat. No. 7,197,354 entitled “System for Determining the Position and Orientation of a Catheter,” and U.S. Pat. No. 7,386,339 entitled “Medical Imaging and Navigation System,” the entire disclosures of which are incorporated herein by reference; a combination electric field-based and magnetic field-based system such as the Carto 3™ System also available from Biosense Webster, and as generally shown with reference to U.S. Pat. No. 7,536,218 entitled “Hybrid Magnetic-Based and Impedance-Based Position Sensing,” the entire disclosure of which is incorporated herein by reference; as well as other impedance-based localization systems, acoustic or ultrasound-based systems, and commonly available fluoroscopic, computed tomography (CT), and magnetic resonance imaging (MRI)-based systems.

In an exemplary embodiment, the catheter 16 includes one or more positioning sensors for producing signals indicative of catheter position and/or orientation information. As will be described in greater detail below, the position and orientation of the catheter 16, and the electrodes 18 thereof, in particular, may be used in the generation of a tissue morphology map. In an embodiment wherein the system 24 is an electric field-based system, the positioning sensor(s) may include one or more of the electrodes mounted in or on the shaft 34 of the catheter 16. For example, in one exemplary embodiment, one or more of the electrodes 18 serve both electrical parameter measuring and position sensing functions. Accordingly, in such an embodiment, because the electrodes 18 are configured serve a dual purpose, the signals produced that are indicative of position and orientation represent the position and orientation of the electrode(s) 18. In other exemplary embodiments, however, the catheter 16 may include certain electrodes dedicated to performing the parameter measuring function, and other electrodes or sensors dedicated to performing the positioning function. In such an embodiment, because the configuration of the catheter 16 is known (e.g., the known spacing between each of the electrodes or sensors mounted on the catheter 16), the signals produced by the positioning sensors combined with the known configuration of the catheter 16 can be used to determine the position and orientation of the parameter measuring electrodes 18.

Alternatively, in an embodiment wherein the system 24 is a magnetic field-based system, the positioning sensor(s) may comprise one or more magnetic sensors configured to detect one or more characteristics of a low-strength magnetic field. For instance, in one exemplary embodiment, the magnetic sensors may comprise magnetic coils disposed on or in the shaft 34 of the catheter 16. In another exemplary embodiment, the magnetic sensor(s) may comprise magnetic materials, such as, for example and without limitation, neodymium, disposed within at least a portion of the shaft 34, thereby rendering the shaft 34 responsive to magnetic fields. In either embodiment, because the configuration of the catheter 16 is known (e.g., the known spacing between the electrodes and sensors mounted on the catheter 16, for example), the combination of the signals produced by the positioning sensor(s) and the known configuration of the catheter 16 can be used to determine the position and orientation of the parameter measuring electrode(s) 18. In another exemplary embodiment, rather than the magnetic sensors being separate from the parameter measuring electrode(s) 18, one or more of the parameter measuring electrodes 18 may comprise magnetic materials, such as, for example and without limitation, neodymium, or elements, such as, for example and without limitation, electromagnets or coils, that render the electrodes 18 responsive to magnetic fields. Accordingly, because the electrodes 18 are configured serve dual purpose of both parameter measuring and position sensing, the signals produced that are indicative of the position and orientation of the magnetic sensor(s) are also indicative of the position and orientation of the parameter measuring electrode(s) 18.

For purposes of clarity and illustration only, the system 24 will hereinafter be described as comprising an electric field-based system, such as, for example, the EnSite NavX™ system identified above. Accordingly, it will be appreciated that while the description below is primarily limited to an embodiment wherein the positioning sensor comprises one or more positioning electrodes, and the electrodes 18 in particular, in other exemplary embodiments, the positioning sensor may comprise one or more magnetic field sensors (e.g., coils) or dedicated positioning electrodes that are separate and distinct from the electrodes 18. Accordingly, visualization, navigation, and mapping systems that include positioning sensors other than the electrodes described below, or electrodes in general, remain within the spirit and scope of the present disclosure.

With continued reference to FIGS. 1 and 4, the system 24 may include a plurality of patch electrodes 48, the ECU 26, and the display device 28, among other components. However, as briefly described above, in another exemplary embodiment, the ECU 26 and/or the display device 28 may be separate and distinct components that are electrically connected to, and configured for communication with, the system 24.

With the exception of the patch electrode 48_{B} called a “belly patch,” the patch electrodes 48 are provided to generate electrical signals used, for example, in determining the position and orientation of the catheter 16, and in the guidance thereof. In one embodiment, the patch electrodes 48 are placed orthogonally on the surface of the body 14 and are used to create axes-specific electric fields within the body 14. For instance, in one exemplary embodiment, patch electrodes 48_{X1}, 48_{X2} may be placed along a first (x) axis. Patch electrodes 48_{Y1}, 48_{Y2} may be placed along a second (y) axis, and patch electrodes 48_{Z1}, 48_{Z2} may be placed along a third (z) axis. Each of the patch electrodes 48 may be coupled to a multiplex switch 50. In an exemplary embodiment, the ECU 26 is configured through appropriate software to provide control signals to switch 50 to thereby sequentially couple pairs of electrodes 48 to a signal generator 52. Excitation of each pair of electrodes 48 generates an electrical field within body 14 and within an area of interest such as tissue 12. Voltage levels at non-excited electrodes 48, which are referenced to the belly patch 48_{B}, are filtered and converted and provided to ECU 26 for use as reference values.

As briefly discussed above, the catheter 16 includes one or more electrodes 18 mounted therein or thereon that are electrically coupled to the ECU 26 and that is/are configured to serve a position sensing function. In an exemplary embodiment, the electrodes 18 are placed within electrical fields created in the body 14 (e.g., within the heart) by exciting the patch electrodes 48. For purposes of clarity and illustration only, the description below will be limited to an embodiment wherein a single electrode 18 is placed within the electric fields. It will be appreciated, however, that in other exemplary embodiments that remain within the spirit and scope of the present disclosure, a plurality of electrodes 18 can be placed within the electric fields and then positions and orientations of each electrode are determined using the techniques described below. When disposed with the electric fields, the electrode 18 experiences voltages that are dependent on the location between the patch electrodes 48 and the position of the electrode 18 relative to tissue 12. Voltage measurement comparisons made between the electrode 18 and the patch electrodes 48 can be used to determine the position of the electrode 18 relative to the tissue 12. Additionally, movement of the electrode 18 proximate the tissue 12 (e.g., within a heart chamber) produces information regarding the geometry of the tissue 12. This information may be used by the ECU 26, for example, to generate models and maps of anatomical structures. Information received from the electrode 18 can also be used to display on a display device, such as display device 28, the location and orientation of the electrode 18 and/or the tip of the catheter 16 relative to the tissue 12. Accordingly, among other things, the ECU 26 of the system 24 provides a means for generating display signals used to the control display device 28 and the creation of a graphical user interface (GUI) on the display device 28.

The ECU 26 may also provide a means for determining the geometry of the tissue 12, EP characteristics of the tissue 12, and the position and orientation of the catheter 16. The ECU 26 may further provide a means for controlling various components of system 10 including, but not limited to, the switch 50 and, as will be described in greater detail below, the tissue sensing circuit 22. It should be noted that while in an exemplary embodiment the ECU 26 is configured to perform some or all of the functionality described above and below, in another exemplary embodiment, the ECU 26 may be separate and distinct from the system 24, and system 24 may have another processor configured to perform some or all of the functionality described herein (e.g., acquiring the position/location of the electrode/catheter, for example). In such an embodiment, the processor of the system 24 would be electrically coupled to, and configured for communication with, the ECU 26. For purposes of clarity and ease of description only, however, the description below will be limited to an embodiment wherein ECU 26 is part of system 24 and configured to perform all of the functionality described herein.

The ECU 26 may comprise a programmable microprocessor or microcontroller, or may comprise an application specific integrated circuit (ASIC). The ECU 26 may include a central processing unit (CPU) and an input/output (I/O) interface through which the ECU 26 may receive a plurality of input signals including, for example, signals generated by patch electrodes 48 and the electrode 18, and generate a plurality of output signals including, for example, those used to control and/or provide data to treatment devices, the display device 28, the switch 50 and the tissue sensing circuit 22. The ECU 26 may be configured to perform various functions, such as those described in greater detail below, with appropriate programming instructions or code (i.e., software). Accordingly, the ECU 26 is programmed with one or more computer programs encoded on a computer storage medium for performing the functionality described herein.

In operation, the ECU 26 generates signals to control the switch 50 to thereby selectively energize the patch electrodes 48. The ECU 26 receives position signals (location information) from the catheter 16 (and particularly the electrode 18) reflecting changes in voltage levels on the electrode 18 and from the non-energized patch electrodes 48. The ECU 26 uses the raw location data produced by the patch electrodes 48 and electrode 18 and corrects the data to account for respiration, cardiac activity, and other artifacts using known or hereafter developed techniques. The ECU 26 may then generate display signals to create an image of the catheter 16 that may be superimposed on an EP map of the tissue 12 generated or acquired by the ECU 26, or another image or model of the tissue 12 generated or acquired by the ECU 26.

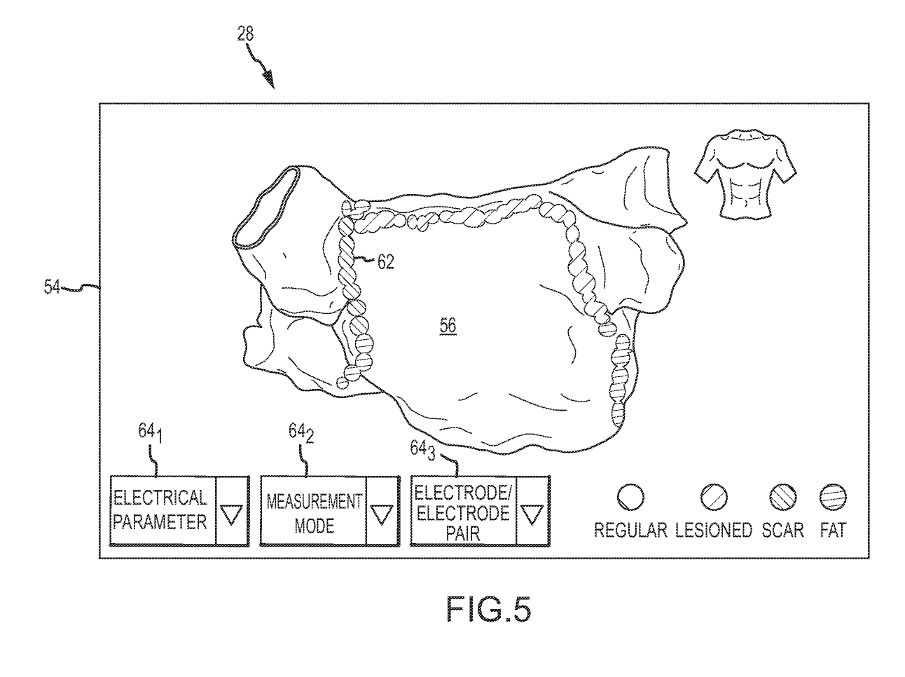

The display device 28, which, as described above, may be part of the system 24 or a separate and distinct component, is provided to convey information to a clinician, such as, for example, information relating to the morphology of the tissue 12. The display device 28 may comprise a conventional computer monitor or other display device known in the art. With reference to FIG. 5, the display device 28 presents a graphical user interface (GUI) 54 to the clinician. The GUI 54 may include a variety of information including, for example and without limitation, an image or model of the geometry of the tissue 12, EP data associated with the tissue 12, electrocardiograms, ablation data associated with the tissue 12, markers corresponding to tissue morphology or tissue type of the tissue 12, electrocardiographic maps, and images of the catheter 16 and/or electrode(s) 18. Some or all of this information may be displayed separately (i.e., on separate screens) or simultaneously on the same screen. As will be described in greater detail below, the GUI 54 may further provide a means by which a clinician may input information or selections relating to various features and functionality of the system 10 into the ECU 26.

The image or model of the geometry of the tissue 12 (image/model 56 shown in FIG. 5) may comprise a two-dimensional image of the tissue 12 (e.g., a cross-section of the heart) or a three-dimensional image of the tissue 12. The image or model 56 may be acquired by the ECU 26 of the system 24 by the ECU 26 generating the image/model 56, or alternatively, the ECU 26 may be configured to obtain image/model 56 that is generated by another imaging, modeling, or visualization system (e.g., fluoroscopic, computed tomography (CT), magnetic resonance imaging (MRI), direct visualization, etc.-based systems). As briefly mentioned above, the display device 28 may also include an image or representation of the catheter 16 and/or the electrode(s) 18 illustrating their position relative to the tissue 12. The image of the catheter 16 may be part of the image 56 itself, or a representation of the catheter 16 and/or electrode 18 may be superimposed onto the image/model 56.

In an exemplary embodiment, and as will be described in greater detail below, the ECU 26 may be further configured generate the GUI 54 on the display device 28 that enables a clinician to enter or otherwise provide various information to the system 10. The information may relate to, for example, the mode of measurement (e.g., unipolar, multi-unipolar, bipolar, multi-bipolar) and/or the particular electrodes for which measurements are desired, electrical parameters to be monitored/measured (e.g., particular components of the complex impedance or other parameters relating to the tissue 12 that the user wants to measure or monitor), visualization schemes to be associated with different tissue types, and criteria to be used in evaluating electrical parameters, such as, for example, the magnitude of evaluation time intervals, the magnitude of various threshold values, and the like.

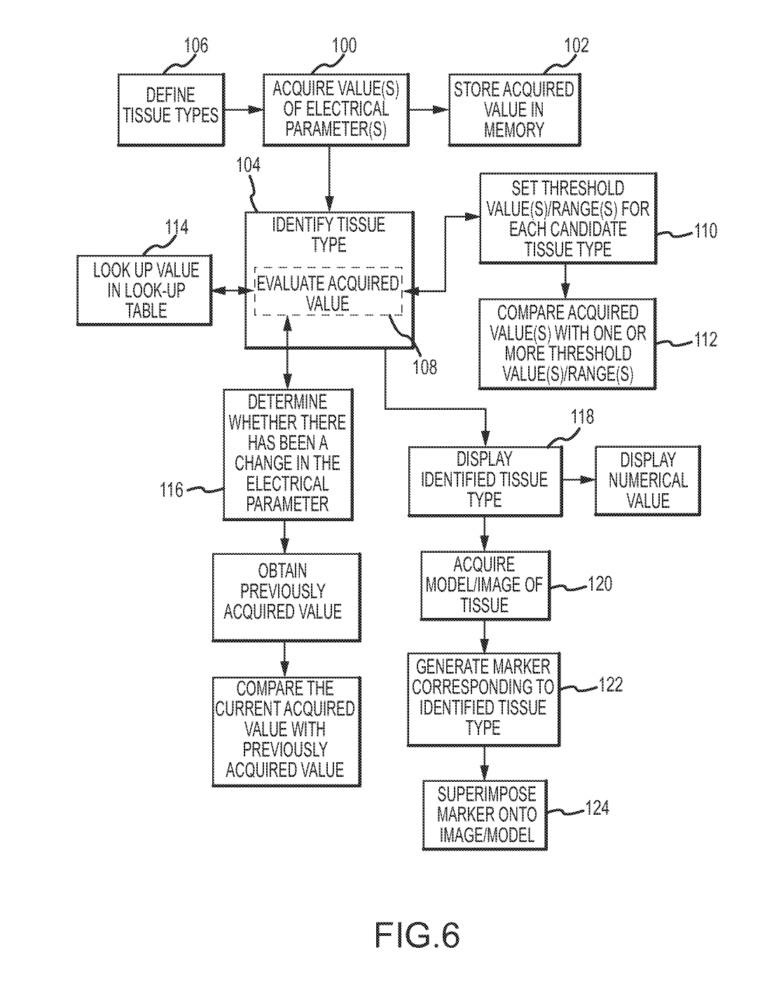

With reference to FIG. 6, in addition to the functionality described above, in an exemplary embodiment, the ECU 26 is further configured to determine tissue type from a plurality of candidate tissue types for one or more locations in the tissue 12 that are, or have been, electrically coupled with one or more of the electrodes 18 of the catheter 16 (e.g., the electrode(s) 18 are or have been in physical contact with the location(s) in the tissue 12, or in sufficient proximity to the location(s) in the tissue 12 such that the electrode(s) is/are electrically coupled therewith). The ECU 26 may be further configured to generate a tissue morphology map that contains representations of one or more tissue type determinations for one or more locations in the tissue 12. It will be appreciated by those of ordinary skill in the art that while the description above and below is directed primarily to an embodiment wherein the ECU 26 performs this functionality, in another exemplary embodiment, the system 10 may include other components (e.g., the tissue sensing circuit 22) or another electronic control unit or processor separate and distinct from the ECU 26 and system 24 that is configured to perform the same functionality in the same manner as that described above and below with respect to the ECU 26. Accordingly, the description wherein the ECU 26 alone is configured to perform this functionality is provided for exemplary purposes only and is not meant to be limiting in nature.

Through experimentation and testing, it has been found that different types of tissue have different characteristics with respect to certain electrical parameters. For example, the reactance of scar tissue is different than that of fat tissue, regular tissue, or ablated/lesioned tissue. Similarly, the impedance magnitude of lesioned tissue is different than that of regular tissue, scar tissue, or fat tissue. Therefore, when the characteristics of certain electrical parameters are known for different tissue types, and measurements of one or more of the certain electrical parameters are made for a particular location in tissue, a determination of the tissue type for that location can be made based on the measured value(s) and the known characteristics of the different tissue types. Accordingly, with continued reference to FIG. 6, and as briefly described above, in an exemplary embodiment, the ECU 26 is generally configured to acquire one or more values of one or more electrical parameters between one of the electrodes 18 electrically coupled with the tissue 12 and another electrode (e.g., another one of the electrodes 18 mounted on the catheter 16 or the patch electrode 20) (step 100), and to identify a tissue type from a plurality of candidate tissue types based on the acquired value(s) (step 104). As will be described in greater detail below, the candidate tissue types may include regular tissue (e.g., endocardial, myocardial, or epicardial tissue), lesioned (i.e., ablated) tissue, ischemic scar tissue, and fat tissue.

The one or more electrical parameters being monitored and for which one or more values are being acquired may comprise any number of electrical parameters. For example, in an exemplary embodiment, and as described in greater detail above, one electrical parameter may be a component of the complex impedance between one of the electrodes 18 (e.g., electrode 18₁) electrically coupled with the tissue 12, and another electrode of the catheter 16 (e.g., the electrode 18₂ also electrically coupled with the tissue 12) or separate and distinct therefrom (e.g., the patch electrode 20 or the electrode of another catheter).

An additional electrical parameter that may be used is an index calculated using one or more components of the complex impedance between an electrode of the catheter 16 (e.g., the electrode 18₁) and another electrode (e.g., the electrode 18₂ or the patch electrode 20, for example). One exemplary index is an electrical coupling index (ECI). It has been found, for example, that the ECI of changed or lesioned (i.e., ablated) tissue is substantially different from that of otherwise similar unchanged (e.g., regular tissue). For example, the ECI of lesioned tissue may be, for example, lower than that of unchanged tissue. Accordingly, as one or more of the electrodes 18 are moved along or across the surface of the tissue 12, ECI calculations are made at a predetermined sampling rate and may be used, as will be described below, to enable a determination as to the type of tissue with which the one or more electrodes 18 are, or were, electrically coupled. One exemplary approach for calculating the ECI is set forth in U.S. Patent Publication No. 2009/0163904, filed May 30, 2008 and entitled “System and Method for Assessing Coupling Between an Electrode and Tissue,” the entire disclosure of which is incorporated herein by reference. To summarize, however, one or more components of the complex impedance measured by, for example, the tissue sensing circuit 22 are acquired by the ECU 26 and used to calculate the ECI using equation (1):

ECI=a*Rmean+b*Xmean+c (1)

wherein “Rmean” is the mean value of a plurality of resistance values, “Xmean” is the mean value of a plurality of reactance values, and a, b, and, c are coefficients dependent upon, among other things, the specific catheter used, the patient, the equipment, the desired level of predictability, the species being treated, and disease states. More specifically, for one particular 4 mm irrigated tip catheter, the ECI is calculated using the equation (2):

ECI=Rmean−5.1*Xmean (2)

Another exemplary index is an ablation lesion index (ALI). In an exemplary embodiment, the ALI is based on the ECI but also takes into account additional confounding factors or parameters not taken into account in the ECI, such as, for example and without limitation, temperature, pressure, contact force, trabeculation, and other parameters. In an embodiment wherein the electrical parameter for which values are acquired is the ALI, the catheter 16 may include additional electrodes/sensors.

One exemplary approach for calculating the ALI is set forth in U.S. Patent Publication No. 2010/0069921, filed Nov. 20, 2009 and entitled “System and Method for Assessing Lesions in Tissue,” the entire disclosure of which is incorporated herein by reference. To summarize, in one exemplary embodiment, the ALI is calculated taking into account ECI, temperature, and force. Accordingly, the ECU 26 is configured to receive inputs comprising components of complex impedance, contact force, and temperature, and to calculate the ALI using, for example, equation (3):

ALI=a₁*ECI+a₂*T+a₃*F (3)

wherein the ECI, T, and F are calculated or measured values of each of the ECI, temperature (T), and contact force (F) at a particular position or location of the tissue at a particular time, and the coefficients a₁, a₂, a₃ are predetermined values intended to account for the dependent relationship between each of the respective variables and other measurements/calculations.

Other electrical parameters may include those that vary with the frequency of the signal injected into the tissue by the signal source 44 (i.e., the electric field to which the tissue is subjected as a result of the injected signal). The frequency characteristics of tissue may be used to infer information about the tissue type, and therefore, may be used to differentiate tissue type. More particularly, different frequencies may be used to create modulation effects of the electric field in the tissue, which subsequently may be used for differentiating tissue and monitoring changes in the tissue based on the modulation characteristics. Accordingly, other electrical parameters that may be used include non-linear electrical properties or parameters that are interrogated using different frequencies of the excitation signal injected into the tissue 12, and therefore, the electric field to which the tissue 12 is subjected. These parameters include, for example and without limitation, the conductivity and permittivity of the tissue.

In one exemplary embodiment, these parameters may be sensed and processed using a discrete frequency technique that may be carried out in either the unipolar or bipolar measurement modes. In such an embodiment, excitation signals of different frequencies are generated by the signal source 44 and applied across the electrodes coupled to the signal source 44 one at a time. For each frequency, a value of the electrical parameter (e.g., the permittivity, conductivity, or the parameters corresponding thereto such as, for example, resistance, reactance, etc.) is determined or sensed by the sensor 46. In an exemplary embodiment, the value of the parameter for each frequency may then be used individually in determining the type of the tissue corresponding to the value in the exemplary manner described below. Alternatively, the values of the parameter for each frequency may be processed together or compared with each other such that a combination of the values may be used in the tissue type determination.

In another exemplary embodiment, which also may find application in one or both of the unipolar and bipolar measurement modes, rather than using signals of discrete frequencies, the signal source 44 is configured to generate a burst signal with a spectrum of frequencies. In such an embodiment, the burst signal is applied across the electrodes coupled to the signal source 44. Using conventional filtering techniques, the tissue sensing circuit 22 may then determine a value of the parameter for one or more of the frequencies within the spectral band to determine the spectral characteristics of the tissue 12. Accordingly, for each desired frequency within the frequency band, a value of the electrical parameter (e.g., the permittivity, conductivity, or the parameters relating thereto such as, for example, resistance, reactance, etc.) may determined or sensed and then used individually or collectively with values corresponding to other frequencies within the spectral band in the tissue type determination.

It will be appreciated that while only a select few exemplary electrical parameters have been specifically identified herein, other known electrical parameters may be acquired and used, and embodiments of the system 10 using these known electrical parameters remain within the spirit and scope of the present disclosure. Further, while in one exemplary embodiment a value of one electrical parameter is acquired by the ECU 26, in other exemplary embodiments values for each of a plurality of electrical parameters may be acquired and used in determining tissue type. For instance, in an exemplary embodiment, multiple components of the complex impedance between the two electrodes may be acquired by the ECU 26 and, as will be described in greater detail below, processed or evaluated together to make a tissue type determination.

The ECU 26 may acquire the value(s) of the monitored electrical parameter(s) in a number of ways. In one exemplary embodiment, the ECU 26 is electrically connected to, and configured for communication with, the tissue sensing circuit 22, which is configured to measure one or more electrical parameters in the manner described in greater detail above. Accordingly, in such an embodiment, the ECU 26 may acquire the values of one or more electrical parameters from the tissue sensing circuit 22. In other exemplary embodiments, however, the ECU 26 may be configured to acquire the value of the electrical parameter from another component of the system 10, or to receive information from the tissue sensing circuit 22, electrodes or sensors of the catheter 16, or other components of the system 10, and to then acquire the value by calculating or resolving the value itself (as in the case with the indices described above). Accordingly, the ECU 26 may acquire the value(s) of the electrical parameter(s) in any number of ways, all of which remain within the spirit and scope of the present disclosure.

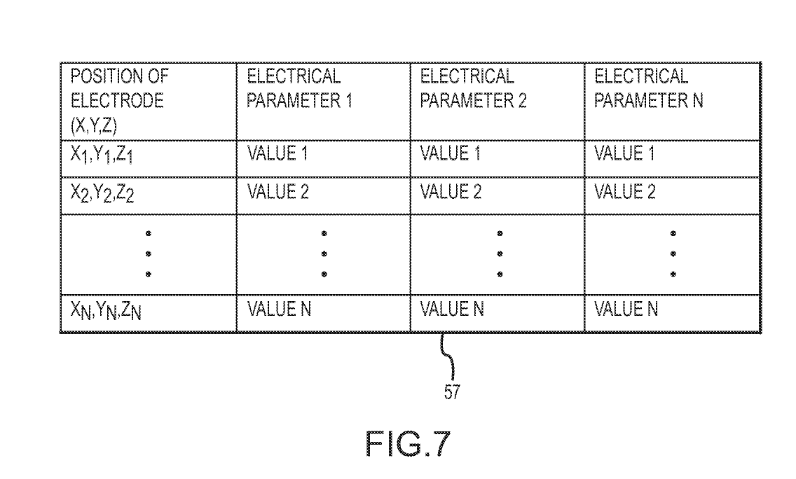

With continued reference to FIG. 6, in an exemplary embodiment, the ECU 26 is configured to store some or all of the acquired values of one or more electrical parameters in, for example, a table 57 (See FIG. 7) stored in a memory or storage device that is part of the ECU 26 or accessible thereby (e.g., the memory 58 depicted in FIG. 1) (step 102). The ECU 26 may be further configured to acquire or determine the position and orientation of the electrode(s) 18 used to measure the electrical parameter value(s) for each acquired and stored parameter value using, for example, the techniques described above with respect to the visualization, navigation, and mapping system 24. Accordingly, in an exemplary embodiment, the ECU 26 is configured to determine, for each acquired and stored parameter, a location in the tissue 12 to which the value(s) correspond (i.e., when a measurement is made, the ECU 26 also determines a position and orientation of the electrode(s) used in the measurement, and therefore, a corresponding location in the tissue 12). As will be described in greater detail below, this information may be used to generate a tissue morphology map for the tissue 12. The ECU 26 may store the position and orientation for each parameter value in the table 57 along with the corresponding parameter value(s).

In view of the fact that a number of electrical parameters may be monitored, acquired, and used as described herein, in an exemplary embodiment, the ECU 26 may be configured to receive instructions from a clinician as to which electrical parameter the clinician would like to monitor. Alternatively, the ECU 26 may be pre-programmed with select electrical parameter(s) to monitor. In an embodiment wherein the clinician can select the electrical parameter(s) to be monitored, the GUI 54 may be configured to provide a means by which the clinician can select one or more electrical parameters to be monitored. The GUI 54 may present an input screen comprising one or more fields in which the clinician may provide his selections. For example, and as illustrated in FIG. 6, the GUI 54 may present the clinician with one or more drop-down menus 64 (e.g., 64₁) that contains a list of electrical parameters that may be monitored. Accordingly, using an input device 60 (See FIG. 1), such as, for example, a mouse, a keyboard, a touch screen or the like, the clinician may select the electrical parameter(s) of interest. In another exemplary embodiment, the GUI 54 may present one or more user-inputtable or selectable fields to allow the clinician to enter or select the desired electrical parameter(s). Alternatively, in certain embodiments, the best or preferred electrical parameter(s) to be used may depend on the type and configuration of the catheter 16 and/or other components of the system 10 being used. In such an embodiment, rather than the clinician selecting the electrical parameter(s) to be used, the catheter 16 may itself include a memory, such as an EEPROM, that stores an identification of the electrical parameters that should be used for that particular catheter and/or other equipment of the system 10, or stores a memory address for accessing the information in another memory location. The ECU 26 may retrieve the information or addresses directly or indirectly and then select the appropriate electrical parameter(s) to be used accordingly.

Further, in addition to, or instead of, allowing the clinician to choose the electrical parameter(s) to be monitored, the GUI 54 may further present the clinician with one or more drop down menus or user-inputtable or selectable fields, such as, for example, drop down menus 64₂ and 64₃ in FIG. 5, to allow the user to enter or select the desired measurement mode (e.g., unipolar, multi-unipolar, bipolar, multi-bipolar) and/or the electrode(s) 18 to be used to measure the electrical parameter(s) of interest. Accordingly, if there is a particular area or location in the tissue 12 the clinician is interested in, for example, the clinician may select the measurement mode and/or the electrode 18 or electrode pair corresponding to that area or location in the tissue to be used to measure the electrical parameter(s) of interest. Similarly, if there is a certain level of specificity or localization the clinician desires, the electrode(s) 18 used in the measurement(s) can be selected to meet the clinician's desires. In such embodiments, the ECU 26 may be configured to exert a measure of control over the tissue sensing circuit 22 to ensure that the measurements being made correspond to the correct electrode(s) 18 or electrode pairs. Alternatively, the ECU 26 may be configured to receive measurements from the tissue sensing circuit 22 corresponding to a plurality of electrodes 18 and/or pairs of electrodes 18, and further configured to reconcile which measurements correspond to which electrodes 18 and/or pairs of electrodes 18, and therefore, which measurements should be used as described below.

Once it is determined which electrical parameter(s) is/are to be monitored, and values for that/those electrical parameter(s) are acquired, the ECU 26 is configured to, among other things, evaluate the value(s) of the parameter(s) (step 108) and to identify the tissue type of the tissue corresponding thereto.

The ECU 26 may evaluate the acquired parameter value(s) in a number of ways to determine and identify the appropriate corresponding tissue type. In an exemplary embodiment, and as illustrated in FIG. 6, the ECU 26 is configured to compare the value(s) to one or more predetermined threshold values or ranges of values corresponding to the various candidate tissue types. More particularly, in an exemplary embodiment, for each electrical parameter being measured/monitored or of interest, a threshold value or range of values is set for each candidate tissue type (step 110), and the ECU 26 is configured to compare the acquired value to each threshold value or range of values (step 112).

In an embodiment wherein the threshold is a single value as opposed to a range of values, each threshold value may be the lowest value of the electrical parameter for that type of tissue, or the highest value of the parameter for that type of tissue. In either instance the ECU 26 is configured to compare the acquired value(s) to one or more of the threshold values and to determine, based on whether the acquired value(s) meets, falls below, or exceeds the threshold value(s), whether the tissue corresponding to the acquired value(s) comprises that particular tissue type. The threshold values may be determined by experimentation and/or analysis performed prior to use of the system 10 (i.e., as part of the manufacturing or set up process, for example).

In an embodiment wherein the threshold comprises a range of values for each tissue type, the ECU 26 is configured to compare the acquired value(s) to one or more of the ranges and to determine, based on whether the acquired value(s) falls within, below, or above the threshold range(s), whether the tissue corresponding to the acquired value(s) comprises that particular tissue type. As with the threshold values described above, the threshold ranges may be determined by experimentation and/or analysis performed prior to use of the system 10 (i.e., as part of the manufacturing or set up process, for example).

In an embodiment wherein multiple electrical parameters, or values the electrical parameter(s) at multiple frequencies, are monitored and values for each are acquired by the ECU 26 and used in the tissue type identification, the same processes described above may be implemented for each electrical parameter or for the electrical parameter at each frequency, and then based on the collective comparisons of the acquired values with the respective thresholds (e.g., threshold value(s) or range(s) of values), a determination can be made as to the tissue type.

Alternatively, rather than comparing the acquired value(s) to threshold values or ranges of values, in an exemplary embodiment illustrated, for example in FIG. 6, the ECU 26 is configured to look up the acquired value(s) in a look-up table stored on, or accessible by, the ECU 26 to determine the tissue type corresponding to the acquired value(s). More particularly, values of the electrical parameter(s) being monitored for each candidate tissue type are stored in a look-up table. When the ECU 26 acquires the measured value(s) of the electrical parameter(s) of interest, it may look up that/those acquired value(s) in the look-up table (step 114) and then determine/identify the tissue type corresponding to the acquired value(s). The values of the electrical parameter(s) for each candidate tissue type may be determined by experimentation and/or analysis performed prior to use of the system 10 (i.e., as part of the manufacturing or set up process, for example).

In another exemplary embodiment, the ECU 26 is configured to determine whether there has been a change in the value of the electrical parameter(s) being monitored (step 116), and to then, based on whether there has been a change, and if so, the nature of the change (e.g., whether the change is positive or negative and/or meets a certain magnitude), determine the tissue type.

For example, in one embodiment, the values of the monitored electrical parameter(s) corresponding to two different locations in the tissue 12 are used to determine whether there has been a change, and if so, the nature of the change. In such an embodiment, the ECU 26 may acquire a value for the electrical parameter being monitored for a first location in the tissue 12 and identify a tissue type based thereon. Then, after a certain amount of time and in accordance with a predetermined sampling rate, or after it has been determined that the catheter 16, and the electrode(s) 18 thereof, in particular, has moved a predetermined distance, the ECU 26 may acquire a subsequent value of the electrical parameter for a second location in the tissue 12. If the first value of the electrical parameter was stored in, for example, the table 57, the ECU 26 may be configured to compare the second or most recent value of the electrical parameter with the previous or first value and determine whether there has been a change, and if so, whether the change is positive or negative, as well as the magnitude of the change. Depending on the nature of the change, if any, the ECU 26 is configured to determine the tissue type of the location in the tissue 12 corresponding to the second or subsequent acquired value of the electrical parameter. For instance, in an exemplary embodiment the electrical parameter of interest is impedance and the first acquired value corresponds to the lesioned tissue type. If it is determined that the impedance has dropped or gone down, the ECU 26 may be configured to identify the tissue type of the location in the tissue 12 corresponding to the second acquired value as scar tissue, since scar tissue has lower impedance than lesioned tissue.

In another embodiment, the values of the monitored electrical parameter(s) for a particular location in the tissue 12 at two different points in time are used to determine whether there has been a change in the monitored electrical parameter(s), and if so, the nature of the change. More specifically, the ECU 26 may acquire a value for an electrical parameter being monitored for a particular location in the tissue 12. The acquired value and the corresponding position and orientation of the electrode(s) 18—which may be acquired from the visualization, navigation, and mapping system 24—are stored in, for example, the memory 58 (e.g., the table 57 stored in the memory 58). Each time the electrode 18 is brought back over that particular location, a subsequent value for the monitored electrical parameter is acquired by the ECU 26. The ECU 26 is configured to then access the prior values corresponding to that particular location from the memory 58 and to compare the current or most recent value with the previous value(s) and determine whether there has been a change, and if so, whether the change is positive or negative, as well as the magnitude of the change. As with the embodiment described above, depending on the nature of the change, if any, the ECU 26 may be configured to determine the tissue type of that particular location in the tissue 12 corresponding to evaluated values of the electrical parameter.

In yet another exemplary embodiment wherein values of the monitored electrical parameter(s) are acquired by the ECU 26 for different excitation signal frequencies, values corresponding to the different frequencies may be compared with each other to determine whether there are differences in the values, and if so, the nature of the differences (e.g., the magnitude of the difference, for example). Depending on whether there are differences and, in certain embodiments, the nature of the differences, the ECU 26 may be configured to determine the tissue type of that particular location in the tissue 12 corresponding to evaluated values of the electrical parameter(s).

In an embodiment wherein multiple electrical parameters are monitored and values for each are acquired and used by the ECU 26 in the tissue type identification, the same processes described above may be implemented for each parameter, and then based on the nature of the collective changes, or lack thereof, in the values, a determination can be made as to the tissue type. For example, if it is determined that the value of one parameter went up and another went down, the collective changes could represent or signify one tissue type.

Accordingly, in view of the above, the changes in or the differences between the values of one or more electrical parameters and, in certain embodiments, the nature of the changes or differences, may be used by the ECU 26 in identifying the tissue type.

Once the ECU 26 identifies a tissue type corresponding to a particular location in the tissue (e.g., the site in the tissue 12 at which the value(s) of the electrical parameter(s) was measured), the ECU 26 may be configured to display the identification in visual form for the clinician to see (step 118). In one exemplary embodiment, the acquired value(s) of the electrical parameter(s) may be displayed in numerical form (e.g., a digital readout) on the display 28 of the visualization, mapping, and navigation system 24.

In another exemplary embodiment, the ECU 26 is configured to generate a tissue morphology map based on the identification made by the ECU 26. More particularly, the identified tissue type may be displayed in concert with the model/image 56 (e.g., 2D or 3D image/model) of the anatomical structure of which the tissue 12 is a part (e.g., the heart or a portion thereof), as well as, in an exemplary embodiment, a real-time representation of the catheter 16 and/or the electrode(s) 18 thereof, on the model or image 56. In an exemplary embodiment, both the representation of the catheter 16 and the image/model 56 may be generated by the ECU 26. However, in another exemplary embodiment, each may be generated by separate and distinct systems that are configured for use in conjunction with each other. In any event, the ECU 26 is configured to acquire the image/model 56 of the tissue 12 (step 120).

Accordingly, with reference to FIGS. 5 and 6, the ECU 26 may be configured to generate a marker, such as markers 62 (e.g., 62₁, 62₂, . . . , 62_{N}), representative of the identified tissue type (step 122), and further configured to superimpose the marker 62 onto a portion of the image/model 56 that corresponds to the location in the tissue 12 at which the acquired value was measured (step 124). The markers 62 may be used in conjunction with any number of visualization schemes to distinguish one tissue type from another. For example, in one exemplary embodiment, the marker 62 is color coded such that a first color represents a first tissue type, a second color represents a second tissue type, and so on. In another exemplary embodiment, rather than color coding the markers 62, different markers (e.g., different shapes, sizes, textures, etc.) are used to differentiate between different tissue types. By placing markers 62 on the image/model 56, a tissue morphology map may be created and presented to the clinician on the display 28.

In order to place the marker 62 in the correct locations, the system 10 may correlate each acquired electrical parameter value with the location in the tissue 12 at which the value was measured. This may be done as described in greater detail above, and therefore the entire description will not be repeated here. To summarize, however, each time a value for the monitored electrical parameter(s) is acquired, a location point is determined, based on the position and orientation of the electrode(s) 18 that measured the acquired value, and correlated with the acquired value. The location point may then be stored in a table, such as the table 57. The ECU 26 may then use the location points to superimpose markers 62 onto the image/model 56 in the correct positions wherein each marker 62 corresponds to, and is representative of, identified tissue types.

The size of the marker 62 that is superimposed onto the image/model 56 may be dependent upon an number of factors. One such factor is the modality or technique used to measure the value of the electrical parameter. For example, in an embodiment wherein the acquired value is measured using the unipolar mode or technique described above, the marker 62 may be substantially the size of the electrode 18 used in the measurement. Alternatively, in an embodiment wherein the acquired value is measured using the bipolar mode or technique described above, the size of the marker 62 may be dependent upon the size and spacing between the electrodes 18 (e.g., 18₁ and 18₂) used in the measurement. Accordingly, the ECU 26 may be configured to generate markers of different sizes.

In addition to the above, in an exemplary embodiment the ECU 26 may compensate for motion occurring within the region in which the catheter 16 is disposed in the generation and placement of the markers 62. Motion may be caused by, for example, cyclic body activities, such as, for example, cardiac and/or respiratory activity. Accordingly, the ECU 26 may incorporate, for example, cardiac and/or respiratory phase into the marker generation and placement.

For example, in one embodiment, the ECU 26 may be configured to employ time-dependent gating in an effort to increase accuracy of the placement of the marker 62. In general terms, time-dependent gating comprises monitoring a cyclic body activity and generating a timing signal, such as an organ timing signal, based on the monitored cyclic body activity. The organ timing signal may be used for phase-based placement, thereby resulting in more accurate tissue morphology mapping throughout the different phases of the cyclic activity.

For the purposes of clarity and brevity, the following description will be limited to the monitoring of the cardiac cycle. It will be appreciated, however, that other cyclic activities (e.g., respiratory activity, combination of cardiac and respiratory activities, etc.) may be monitored in similar ways and therefore remain within the spirit and scope of the present invention. Accordingly, in an exemplary embodiment, the system 10 includes a mechanism to measure or otherwise determine a timing signal of a region of interest of the patient's body, which, in an exemplary embodiment, is the patient's heart, but which may also include any other organ that is being evaluated. The mechanism may take a number of forms that are generally known in the art, such as, for example, a conventional electro-cardiogram (ECG) monitor. A detailed description of a ECG monitor and its use/function can be found with reference to U.S. Patent Publication No. 2010/0168550, filed Dec. 31, 2008 and entitled “Multiple Shell Construction to Emulate Chamber Contraction with a Mapping System,” which is incorporated herein by reference in its entirety.

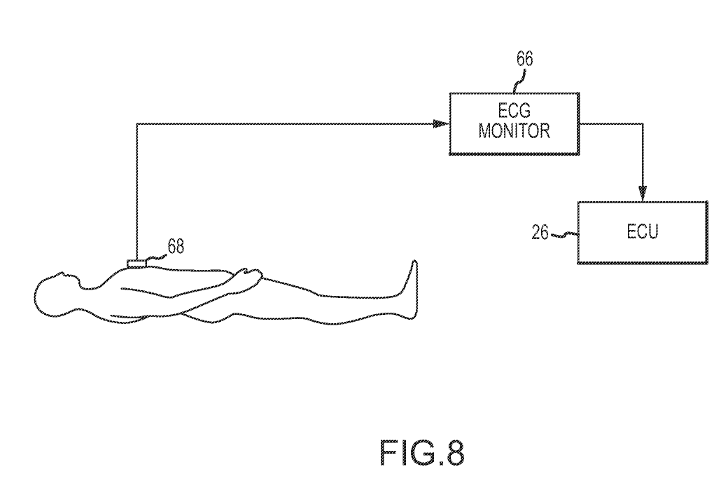

With reference to FIG. 8, in general terms, an ECG monitor 66 is provided that is configured to continuously detect an electrical timing signal of the patient's heart through the use of a plurality of ECG electrodes 68, which may be externally-affixed to the outside of a patient's body. The timing signal generally corresponds to the particular phase of the cardiac cycle, among other things. In another exemplary embodiment, rather than using an ECG to determine the timing signal, a reference electrode or sensor positioned in a fixed location in the heart may be used to provide a relatively stable signal indicative of the phase of the heart in the cardiac cycle (e.g., placed in the coronary sinus). In still another exemplary embodiment, a medical device, such as, for example, a catheter having an electrode may be placed and maintained in a constant position relative to the heart to obtain a relatively stable signal indicative of cardiac phase. Accordingly, one of ordinary skill in the art will appreciate that any number of known or hereinafter developed mechanisms or techniques, including but not limited to those described above, may be used to determine a timing signal.

Once the timing signal, and therefore, the phase of the patient's heart, is determined, the position information corresponding to the electrode(s) 18, and therefore, the electrical parameter values corresponding to the position information, may be segregated or grouped into a plurality of sets based on the respective phase of the cardiac cycle during or at which each position was collected. Once the position and electrical parameter information is grouped, the ECU 26 is configured to generate a tissue morphology map for one or more phases of the cardiac cycle comprising markers 62 representing tissue types determined during each respective phase of the cycle. Because the timing signal is known, as each subsequent position of the electrode 18 and values for the electrical parameter(s) corresponding to that position are acquired, the position and parameter values are tagged with a respective time-point in the timing signal and grouped with the appropriate previously recorded position and parameter information. The subsequent positions and values may then be used to generate tissue morphology maps for the phase of the cardiac cycle during which the position and parameter values were collected/acquired.

Once a tissue morphology map is generated for each phase of the cardiac cycle, the tissue morphology map corresponding to the current phase of the timing signal may be presented to the user of the system 10 at any time. In an exemplary embodiment, the ECU 26 may be configured to play-back the tissue morphology maps (e.g., sequentially reconstructed and displayed on the display 28) in accordance with the real-time measurement of the patient's ECG. Therefore, the user may be presented with an accurate real-time tissue morphology map regardless of the phase of the cardiac cycle. Accordingly, it will be understood and appreciated that the tissue morphology map for each phase may be stored in a memory or storage medium, such as, for example, the memory 58, that is either part of or accessible by the ECU 26 such that the ECU 26 may readily obtain, render, and/or display the appropriate tissue morphology map.

In another exemplary embodiment, rather than the ECU 26 generating a tissue morphology map or otherwise providing an indication as to the identified tissue type, the ECU 26 may be configured to generate electrical signals corresponding to, and representative of, the identification made by the ECU 26, and to send the signals to another component within the system 10 where they may be used in the generation of a tissue morphology map or to otherwise provide the clinician an indication of the tissue type.

As described in greater detail above, in exemplary embodiments, multi-unipolar or multi-bipolar electrical parameter measuring modes may be utilized to acquire values of one or more electrical parameters of interest for a plurality of locations in the tissue 12 substantially simultaneously. In such embodiment, the description above with respect to the identification of tissue type, the generation of a tissue morphology map, and the generation and provision of electrical signals corresponding to, and representative of, identifications made by the ECU 26, apply to these embodiments with equal force. More particularly, for each location in the tissue for which a value(s) of the electrical parameter(s) of interest is/are acquired, the ECU 26 may be configured to perform the tissue type identification, morphology map generation, and/or signal generation. In an exemplary embodiment, the ECU 26 may be configured to carry out the functionality for each location simultaneously. Accordingly, embodiments of the system 10 that perform the functionality described herein for one location in the tissue 12 at a time, and embodiments of the system 10 that perform the functionality described herein for multiple locations in the tissue 12 simultaneously, both remain within the spirit and scope of the present disclosure.

In addition to the above, in an exemplary embodiment, the values acquired for one or more of the above-described electrical parameters using the techniques described above may be used for purposes in addition to or instead of the determination of tissue type and the generation of a tissue morphology map. For example, the values may be used in a lesion formation algorithm to assess lesion formation in the tissue, and/or used to predict the amount of ablative energy being directed to the tissue for use in a lesion formation algorithm. An exemplary approach of assessing lesion formation with which the values acquired using the techniques described herein may be used is set forth in U.S. Patent Application Serial No. 12/946,941, filed Nov. 16, 2010 and entitled “System and Method for Assessing the Formation of a Lesion in Tissue,” the entire disclosure of which is incorporated herein by reference.

It will be appreciated that in addition to the structure of the system 10 described above, another aspect of the present disclosure is a method for determining tissue type and, in an exemplary embodiment, presenting information representative of determined tissue type. In an exemplary embodiment, and as described above, the ECU 26 of the system 10 is configured to perform the methodology. However, in other exemplary embodiments, the ECU 26 is configured to perform some, but not all, of the methodology. In such an embodiment, another component of the system 10 or another electronic control unit or processor that is part of the system 10, or that is configured for communication with the system 10, and the ECU 26 thereof, in particular, is configured to perform some of the methodology.

In either instance, and with reference to FIG. 6, in an exemplary embodiment the method includes a step 100 of acquiring a value(s) of one or more electrical parameters between a pair of electrodes wherein at least one of the electrodes is electrically coupled with tissue 12. In one exemplary embodiment, the one or more electrical parameters for which one or more value(s) are acquired comprises one or more components of the complex impedance between the electrodes. In an exemplary embodiment, the pair of electrodes comprise one of the electrodes 18 of the catheter 16 and one of the indifferent/dispersive electrode 20 affixed to the patient. In another exemplary embodiment, however, the pair of electrodes comprises two of the electrodes 18 of the catheter 16. In yet another exemplary embodiment, the pair of electrodes comprise one of the electrodes 18 of the catheter 16, and an electrode of another catheter used in conjunction with the catheter 16. Further, in an exemplary embodiment, and prior to the acquiring step 100, the method comprises a step of determining or discerning which measurement mode and/or which electrode(s) 18 or pair(s) of electrodes are to be used in the acquisition of the value(s) of the monitored electrical parameter(s). The determination may be made in response to a user input corresponding to the desired mode and/or electrode(s) or in accordance with a predetermined or pre-programmed instruction.

In an exemplary embodiment, the step 100 comprises acquiring values of one or more electrical parameters between each electrode pair of a plurality of electrode pairs. In an exemplary embodiment, the electrical parameters for which values are acquired are the same for each electrode pair. However, in another exemplary embodiment, the electrical parameters for which values are acquired may be different for different electrode pairs. In an exemplary embodiment, the plurality of electrode pairs each comprise an electrode 18 of the catheter 16 and the indifferent/dispersive electrode 20 affixed to the patient. Alternatively, in another exemplary embodiment, the plurality of electrode pairs each comprise two electrodes 18 of the catheter 16. In one exemplary embodiment, one of the electrodes 18 of the catheter 16 may be common to each electrode pair. However, in another exemplary embodiment, none of the electrodes of the electrode pairs may be common to each other. In an embodiment wherein values of one or more electrical parameters are acquired for a plurality of electrode pairs, a value of a single electrical parameter may be acquired or values for a plurality of electrical parameters may be acquired. Further, in an exemplary embodiment, the values for each electrode pair may be acquired simultaneously.

In any of the embodiments described above, the method may further comprise a step 102 of storing the acquired value(s) of the electrical parameter(s) in a table, for example, of a memory or storage device.

In an exemplary embodiment, the method further comprises a step 104 of identifying a tissue type from a plurality of candidate tissue types based on at least the acquired value(s) of the electrical parameter(s). In an embodiment wherein a value of a single electrical parameter is acquired, the identifying step 104 comprises identifying a tissue type for the tissue proximate the electrode pair based on that single value. In an embodiment wherein values of a plurality of electrical parameters are acquired, the identifying step 104 may comprise identifying a tissue type for the tissue proximate the electrode pair based on one or more of the acquired values. In an embodiment wherein values of one or more electrical parameters are acquired for a plurality of electrode pairs, the identifying step may comprise identifying a tissue type for the tissue proximate one or more of the electrode pairs based on one or more of the corresponding acquired values.

In an exemplary embodiment, the method further comprises the step 106 of defining candidate tissue types. In an exemplary embodiment, the step 106 comprises defining the tissue types to include regular tissue (e.g., endocardial, myocardial, or epicardial tissue), lesioned tissue, ischemic scar tissue, and fat tissue. It will be appreciated, however, that in other exemplary embodiments, other types of tissue may be used instead of or in addition to those specifically identified herein. Step 106 may be performed either prior to the step 100 of acquiring the parameter value(s) or after.

In an exemplary embodiment, the identifying step 104 may further include a substep 108 of evaluating the acquired value(s) of the electrical parameter(s) in order to identify the tissue type of the tissue corresponding thereto.

In an exemplary embodiment, the evaluating step 108 comprises comparing the value(s) to one or more predetermined threshold values or ranges of values corresponding to the various candidate tissue types. More particularly, in an exemplary embodiment, the evaluating step 108 comprises a first substep 110 of setting or defining threshold value(s) or range of values for each electrical parameter for each candidate tissue type, and a second substep 112 of comparing the acquired value(s) to one or more of the threshold values or threshold value ranges. Based on the comparison(s), a tissue type for the location in the tissue corresponding to the acquired value(s) may be identified.

In another exemplary embodiment, rather than comparing the acquired value(s) to threshold values or ranges of values, the evaluating step 108 comprises a substep 114 of looking up the acquired value(s) in a look-up table to determine the tissue type of the location in the tissue corresponding to the acquired value(s). More particularly, values of the electrical parameter(s) being monitored for each candidate tissue type are stored in a look-up table. When a value of an electrical parameter is acquired, the acquired value may be looked up in the look-up table and then a tissue type may be identified that corresponds to the acquired value.

In another exemplary embodiment, rather than comparing acquired value(s) to threshold values or ranges, or looking up the acquired value(s) in a look-up table, the evaluating step 108 comprises a sub step 116 of determining whether there has been a change in the value of the electrical parameter(s) being monitored, and then, based on whether there has been a change, and if so, whether the change is positive or negative and/or meets a certain magnitude, determining or identifying the tissue type. A description of an exemplary technique for determining whether there has been a change in the value of the electrical parameter(s) being monitored, and then, based on whether there has been a change and the nature of the change, determining or identifying tissue type, is set forth in great detail above, and therefore, will not be repeated here.

In an exemplary embodiment, the method further includes a step 118 of displaying the identification of tissue type in visual form for the clinician to see. In one exemplary embodiment, the step 118 comprises displaying the acquire value(s) in numerical form (e.g., a digital readout) on a display device.

In another exemplary embodiment, the step 118 comprises generating a tissue morphology map based on the identification of tissue type. More particularly, the identified tissue type may be displayed in concert with a model/image of the anatomical structure of which the tissue is a part (e.g., the heart or a portion thereof). In an exemplary embodiment, the step 118 comprises a substep 120 of acquiring the image/model of the tissue (e.g., generating the image/model or obtaining it from another component). The step 118 further comprises a substep 122 of generating a marker representative of the identified tissue type, and a substep 124 of superimposing the marker onto a portion of the image/model that corresponds to the location in the tissue at which the acquired value was measured. The markers may be used in conjunction with any number of visualization schemes to distinguish one tissue type from another. For example, in one exemplary embodiment, the marker is color coded such that a first color represents a first tissue type, a second color represents a second tissue type, and so on. In another exemplary embodiment, rather than color coding the markers, different markers (e.g., different shapes, sizes, etc.) are used to differentiate between different tissue types. By placing markers on the image/model, a tissue morphology map may be created and presented to the clinician on a display device.

In order to place the marker in the correct locations, the step 118 may further comprise a substep of correlating each acquired electrical parameter value with the location in the tissue at which the value was measured. This may be done as described in greater detail above, and therefore the entire description will not be repeated here. To summarize, however, each time a value for the monitored electrical parameter(s) is acquired, a location point is determined, based on the position and orientation of the electrode that measured the value of the electrical parameter, and correlated with the acquired value. The location points may then be used to superimpose markers onto the image/model in the correct positions wherein each marker corresponds to, and is representative of, an identified tissue type.

It will be appreciated that additional functionality described in greater detail above with respect to the system 10 may also be part of the inventive methodology. Therefore, to the extent such functionality has not been expressly described with respect to the methodology, the description thereof above is incorporated herein by reference.

It should be understood that the system 10, and particularly the ECU 26, as described above may include conventional processing apparatus known in the art, capable of executing pre-programmed instructions stored in an associated memory, all performing in accordance with the functionality described herein. It is contemplated that the methods described herein, including without limitation the method steps of embodiments of the invention, will be programmed in a preferred embodiment, with the resulting software being stored in an associated memory and where so described, may also constitute the means for performing such methods. Implementation of the invention, in software, in view of the foregoing enabling description, would require no more than routine application of programming skills by one of ordinary skill in the art. Such a system may further be of the type having both ROM, RAM, a combination of non-volatile and volatile (modifiable) memory so that the software can be stored and yet allow storage and processing of dynamically produced data and/or signals.

Although only certain embodiments have been described above with a certain degree of particularity, those skilled in the art could make numerous alterations to the disclosed embodiments without departing from the scope of this disclosure. Joinder references (e.g., attached, coupled, connected, and the like) are to be construed broadly and may include intermediate members between a connection of elements and relative movement between elements. As such, joinder references do not necessarily infer that two elements are directly connected/coupled and in fixed relation to each other. Additionally, the terms electrically connected and in communication are meant to be construed broadly to encompass both wired and wireless connections and communications. It is intended that all matter contained in the above description or shown in the accompanying drawings shall be interpreted as illustrative only and not limiting. Changes in detail or structure may be made without departing from the invention as defined in the appended claims.

What is claimed is:

1. A method of determining tissue type comprising the steps of:
acquiring a value of an electrical parameter between a first electrode electrically coupled with tissue and a second electrode; and
identifying a tissue type from a plurality of tissue types based on at least said acquired value of said electrical parameter.

2. The method of claim 1 wherein said electrical parameter comprises at least one component of a complex impedance between said first and second electrodes.

3. The method of claim 1 wherein said acquiring step comprises acquiring a value for each of a plurality of electrical parameters between said first and second electrodes, and said identifying step comprises identifying said tissue type based on said acquired values of said plurality of electrical parameters.

4. The method of claim 1 further comprising defining the plurality of tissue types to include regular, lesioned, scar, and fat.

5. The method of claim 1 wherein said second electrode is a dispersive/indifferent reference electrode, and said acquired value of said electrical parameter reflects properties of tissue proximate said first electrode.

6. The method of claim 1 wherein said first and second electrodes are each electrodes electrically coupled with the tissue, and said acquired value of said electrical parameter reflects properties of tissue between said first and second electrodes.

7. The method of claim 6 wherein said first and second electrodes are mounted on a catheter, and said acquired value of said electrical parameter reflects properties of tissue between said first and second electrodes

8. The method of claim 1 further comprising the step of acquiring a value of an electrical parameter between a third electrode electrically coupled with tissue and one of said first electrode, said second electrode, and a fourth electrode, and wherein said identifying step comprises identifying at least one tissue type based on said values of said electrical parameters between first and second electrodes, and said third and said one of said first, second and fourth electrodes.

9. The method of claim 8 wherein said steps of acquiring said values of said electrical parameters between said first and second electrodes, and said third and said one of said first, second and fourth electrodes are performed substantially simultaneously.

10. The method of claim 1 wherein said identifying step comprises comparing said value to at least one predetermined threshold value of said electrical parameter corresponding to one of said plurality of tissue types.

11. The method of claim 1 wherein said identifying step comprises looking up said value in a look-up table.

12. The method of claim 1 wherein said identifying step comprises determining a change in the value of said electrical parameter over a predetermined period of time.

13. The method of claim 1, further comprising the step of generating a tissue morphology map based on said identified tissue type.

14. The method of claim 1 wherein the tissue is epicardial tissue of a heart.

15. A system for determining tissue type, comprising
an electronic control unit (ECU) configured to:
acquire a value of an electrical parameter between a first electrode electrically coupled with tissue and a second electrode; and
identify a tissue type from a plurality of tissue types based on at least said acquired value of said electrical parameter.

16. The system of claim 15 wherein said electrical parameter comprises at least one component of a complex impedance between said first and second electrodes.

17. The system of claim 15 wherein said ECU is configured to acquire a value for each of a plurality of electrical parameters between said first and second electrodes, and to identify said tissue type based on said acquired values of said plurality of electrical parameters.

18. The system of claim 15 wherein said ECU is configured to:
acquire a value of an electrical parameter between a third electrode electrically coupled with tissue and one of said first electrode, said second electrode, and a fourth electrode; and
identify at least one tissue type based on said values of said electrical parameters between said first and second electrodes, and said third and said one of said first, second and fourth electrodes.

19. The system of claim 18 wherein said ECU is configured to acquire said values of said electrical parameters between said first and second electrodes and said third and said one of said first, second, and fourth electrodes substantially simultaneously.

20. The system of claim 15 wherein said ECU is further configured to generate a tissue morphology map based on said identified tissue type.

21. A method of presenting information representative of determined tissue type comprising the steps of:
acquiring a value of an electrical parameter between a first electrode electrically coupled with tissue and a second electrode;
identifying a tissue type from a plurality of tissue types based on at least said acquired value of said electrical parameter;
determining a location in the tissue corresponding to said acquired value based on a position of said first electrode;
generating a marker representative of the identified tissue type; and
superimposing said marker onto a portion of an image or model of the tissue corresponding to said location.
